(19) United States
(12) Patent Application Publication
(10) Pub. No.: US 2023/0321114 A1
(43) Pub. Date: Oct. 12, 2023

(54) METHODS OF TREATING AGE-RELATED COGNITIVE DECLINE

(71) Applicant: Woolsey Pharmaceuticals, Inc., St. Petersburg, FL (US)

(72) Inventors: Thomas MACALLISTER, Arlington, VA (US); Sven JACOBSON, New York, NY (US)

(21) Appl. No.: 18/042,785

(22) PCT Filed: Jan. 8, 2021
(86) PCT No.: PCT/US2021/012597

§ 371 Date: Feb. 24, 2023

Related U.S. Application Data
(60) Provisional application No. 63/071,259, filed on Aug. 27, 2020.

(51) Int. Cl. A61K 31/551

(52) U.S. Cl. CPC A61K 31/551

(57) ABSTRACT

Methods of treating subjects with age-related cognitive decline with a rho kinase inhibitor are disclosed. In a preferred embodiment, the rho kinase inhibitor is fasudil and it is administered orally in a daily dose of between 70 and 250 mg per day. Subjects have a cognitive impairment on a global cognitive scale, like the MoCA or the MMSE and/or specific impairments related to different cognitive domains. A method of reducing the rate of age-related cognitive decline, comprising administering to a subject with evidence of age-related cognitive decline an effective amount of a rho kinase inhibitor. The inventive methods slow the rate of cognitive decline and/or improve cognition from baseline in treated individuals. The methods may result in enhancing processing speed, increasing attention, improving memory, improving language, improving visual construction skills and/or improving executive function in subjects experiencing age-related cognitive decline.

### CROSS-REFERENCE TO RELATED APPLICATIONS

This application claims priority to U.S. provisional application No. 63/071,259, filed on Aug. 27, 2020, the disclosure of which is incorporated herein in its entirety.

### BACKGROUND OF THE INVENTION

Cognitive decline is part of the normal process of aging and it has been well documented. Some cognitive abilities like vocabulary are generally resistant to this decline; conceptual reasoning, memory, and processing speed, are not and they decline gradually over time. The rate of decline varies widely among the older population. As in the diseased population, declining cognitive skills in normal aging can interfere with activities of daily living and adversely affect the ability to live independently.

In defining age-related cognitive decline, cognitive abilities are divided into those that change with age—so-called fluid abilities—and which do not—so-called crystallized abilities. Crystallized abilities refer to the cumulative skills and memories that result from past cognitive processing. Thus, abilities relating to general knowledge, historical information, and vocabulary, for example, would be considered crystallized abilities. Fluid abilities, in contrast, require present cognitive processing and so manipulation and transformation of information would be required to complete an assessment of fluid abilities. Tests of fluid abilities also require the subject to perceive and adapt to their environment and process new information to solve problems. Crystallized abilities continue to improve throughout life until approximately age 60; fluid abilities begin declining at around age 20.

Cognitive domains affected by aging include attention, memory, executive cognitive function, language, and visuospatial abilities. Cognitive performance requires the subject to perceive a stimulus, process the information, and then respond. Thus, decreased sensory perception can confound measures of cognition. Auditory acuity, speech discrimination and sound localization, for example, are known to decline with age. Hearing loss ? begins to decline after age 30, and up to 70% of subjects age 80 have measurable hearing loss. Underlying declining cognitive performance on timed tests, which may touch on a number of different cognitive domains, is an age-related decline in processing speed.

Some of the most noticeable age-related changes are reflected in declines in performance on complex attentional tasks involving selective or divided attention. Selective attention is the ability to focus on relevant information while ignoring irrelevant information in a complex environment. Divided attention refers to the ability to focus on multiple things at the same time.

New learning abilities decline with increasing age, along with the ability to retrieve newly learned material. New learning, as measured by delayed free recall, declines with age. Working memory, which requires active manipulation of material to be learned, also declines with age. Prospective memory, remembering to perform intended action in the future also declines with age.

Executive cognitive function also declines with age. Affected domains include decision making, problem solving, planning and sequencing of responses, and multitasking. Executive function is important for novel tasks that cannot be addressed habitually. Thus, performance on tests that are novel, complex, or timed steadily declines with advancing age, as does performance on tests that require selectively inhibiting some responses or tests that involve distinguishing relevant and irrelevant information. Concept formation, abstraction, and mental flexibility also decline with age.

With respect to language, speech comprehension in the setting of background noise and ambiguous speech content declines with age. Verbal fluency, verbal retrieval, and some confrontational naming tasks also decline with age.

Visuospatial processing and constructional praxis also decline with age. Specifically, visuoperceptual judgment and ability to perceive spatial orientation become impaired with advancing age. (Salthouse, 2009)

There is one report in the literature relating to the use of fasudil in the context of normal aging. Huentelman (2009) examined the effects of the fasudil derivative hydroxyfasudil on memory in aging rats. Eighteen-month old rats were treated with vehicle, 0.1875 mg per day of hydroxyfasudil or 0.3750 mg per day of hydroxyfasudil. Drug was administered in the morning prior to testing as a single daily dose by injection, though it is not specified whether the route of intravenous (iv), subcutaneous (sc), intraperitoneal (ip) or some more exotic route. Treatment was begun 4 days prior to behavioral testing and continued throughout the testing period. Animals were tested in two versions of the Morris Water Maze—the standard maze, which is a measure of spatial reference memory, and the radial maze, which can measure both spatial reference memory and working memory. The authors reported improvements in working memory, but not in spatial reference memory.

While interesting, it is difficult to extrapolate from Huentelman work to humans. First, the extrapolation from animals to humans is always difficult and there are more examples of animal studies that do not translate to humans than there are that do translate. Second, it is unclear how to extrapolate to a usable dose. The authors used hydroxyfasudil, which has never been developed as a systemic drug in humans. While it is a metabolite of fasudil, which has been studied in humans, it is unclear how the rat metabolism affects its activity relative to the parent molecule. Moreover, the differential activities of the parent versus the metabolite are not known, and when the parent is administered, there will be a mixture of compounds circulating, whereas in this experiment clearly it is only the metabolite. Compounding this is that the authors do not disclose the route of delivery. Whether it was administered iv, ip, sc or another route invokes different metabolic routes that can affect the activity of the drug—it may become more or less active or more or less bioavailable. Without further information, this cannot be assessed. Finally, any observations from this paper are limited to improvements in working memory and do not at all relate to the many other domains of cognition that are affected by aging.

Currently, there are no effective therapeutic approaches to treating age-related cognitive decline. There is a very active supplement industry attempting to address this issue, but all of them lack any scientific rigor and remain unapproved by regulators. At present, the widespread interventions limited to lifestyle choices like increased physical activity, mental stimulation, avoiding excessive exposure to neurotoxins (e.g., alcohol), treating depression and managing stress, and controlling common medical conditions such as hypertension, diabetes, and obstructive sleep apnea. There exists a significant unmet need to provide new, approaches to mitigating or reversing age-related cognitive decline.

### SUMMARY OF THE INVENTION

The invention contemplates the treatment of age-related cognitive decline. In a preferred embodiment, the rho kinase inhibitor is fasudil and it is administered orally in a daily dose of between 70 and 250 mg per day. In preferred methods, subjects have a cognitive impairment on a global cognitive scale, like the MoCA or the MMSE and/or specific impairments related to different cognitive domains. Some embodiments treat subjects with reduced verbal fluency, declarative memory and/or semantic memory. For patients with memory deficits, the invention contemplates treating subjects suffering from a reduced rate of memory acquisition or a reduced ability to retrieve memories.

The inventive methods further contemplate treating subjects with language deficits and, in particular, those who show a deficit in visual confrontation naming and/or verbal fluency. Improving deficits in visual construction skills are also within the scope of the invention.

Treating subjects with deficits in executive function is also contemplated, especially when the subject has a deficiency in one or more of the following abilities: concept formation, abstraction, mental flexibility, response inhibition, speed-motor executive functions, inductive reasoning and reasoning with unfamiliar information.

In one embodiment, treatment with fasudil results in improved fluid intelligence compared to that measured in the same subject prior to treatment with fasudil or in a longitudinal cohort of subjects.

In another embodiment, treatment with fasudil results in improved sustained and/or selective attention compared to that measured in the same subject prior to treatment with fasudil or in a longitudinal cohort of subjects.

In a further embodiment, treatment with fasudil results in improved declarative, i.e., episodic and/or semantic memory, compared to that measured in the same subject prior to treatment with fasudil or in a longitudinal cohort of subjects.

In a specific embodiment, treatment with fasudil results in improvements in delayed free recall, i.e., spontaneous retrieval of information from memory without a cue (e.g., a grocery list).

In another specific embodiment, treatment with fasudil result in improvements in prospective memory, i.e., remembering to perform intended actions in the future such as taking medicine before bed.

In another embodiment, treatment with fasudil results in improved language ability compared to that measured in the same subject prior to treatment with fasudil or in a longitudinal cohort of subjects.

In yet another embodiment, treatment with fasudil result in improvement in processing speed and executive function.

In another embodiment, treatment with fasudil result in improvement in visual construction skills such as putting furniture together.

In a further embodiment, treatment with fasudil results in improvement in the speed of motor abilities (e.g., driving).

In one embodiment, treatment with fasudil results in reduced or delayed gray matter loss and/or improves synaptic density in the prefrontal cortex compared to that measured in the same subject prior to treatment with fasudil or in a longitudinal cohort of subjects.

In another embodiment, treatment with fasudil results in reduced or delayed white matter loss compared to that measured in the same subject prior to treatment with fasudil or in a longitudinal cohort of subjects.

In one embodiment, subject treated with fausdil is aged 60 or older. In another embodiment, the subject treated with fausdil is 40-59.

In further embodiment, the subject treated with fausdil is aged 20-39.

In one embodiment, the subject treated with fasudil has a lower than average childhood IQ.

In one embodiment, the subject treated with fausdil does not have mild cognitive impairment.

In another embodiment, the subject treated with fausdil possesses the E4 allele of the APOE gene

In a further embodiment, the subject treated with fausdil has vascular disease including but not limited to high blood pressure, artherosclerosis impaired ventricular function, and peripheral arterial disease.

in another embodiment, the subject treated with fausdil exhibits increased levels of inflammatory oxidative markers (C-reactive protein, TNF-alpha IL-6)

In a further embodiment, the the subject treated with fausdil has no post-secondary education.

In a specific embodiment, the invention contemplates co-administration of fasudil in combination with B-vitamins including B12, B6, B9, antioxidants (e.g., vitamins, C, E, beta-carotene and flavonoids), and omega 3-fatty acids;

In another specific embodiment, the invention contemplates co-administration of fasudil in combination with plant polyphenols such as resveratrol.

### DETAILED DESCRIPTION OF THE INVENTION

The invention is based on the discovery that rho kinase inhibitors can be used to treat age-related cognitive decline. As used herein, age-related cognitive decline is defined as the decline in cognition that occurs as a result of the normal aging process and not due to a pathological process. Cognition is defined as the mental action or process of acquiring knowledge and understanding through thought, experience, and the senses. Cognition encompasses attention, the formation of knowledge, memory and working memory, judgment and evaluation, reasoning and computation, problem-solving and decision-making, comprehension and production of language. Cognitive processes use existing knowledge and generate new knowledge. Thus, the deficiencies addressable according to the invention may encompass any of the foregoing aspects of cognition that decline with advancing age.

### ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p60ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinoline derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors include indazole, pyrimidine, pyrrolopyridine, pyrazole, benzimidazole, benzothiazole, benzathiophene, benzamide, aminofurazane, quinazoline, and boron derivatives (Feng et al., 2015). Some exemplary ROCK inhibitors are shown below:

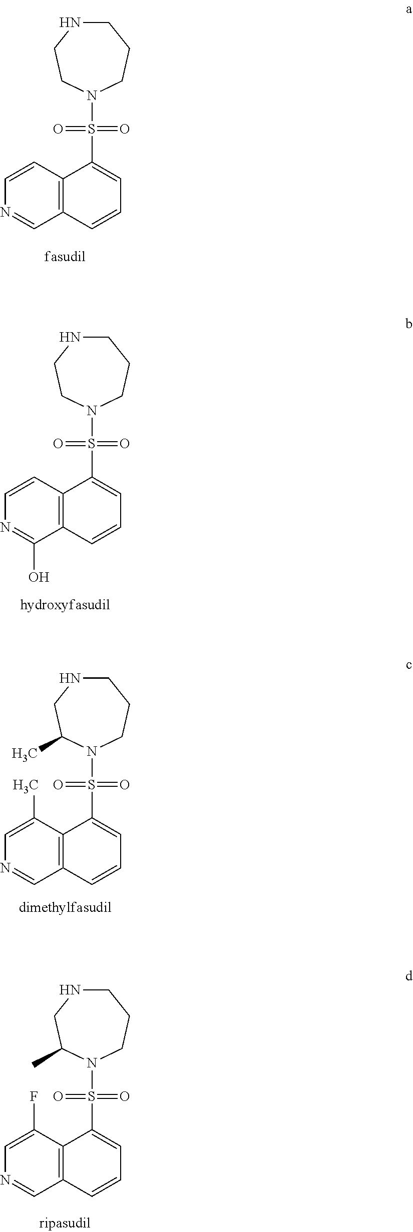 [SMILES: O=S(=O)(c1cccc2cnccc12)N1CCCNCC1 ; O=S(=O)(c1cccc2c(O)nccc12)N1CCCNCC1 ; Cc1cncc2cccc(S(=O)(=O)N3CCCNC[C@@H]3C)c12 ; C[C@H]1CNCCCN1S(=O)(=O)c1cccc2cncc(F)c12]

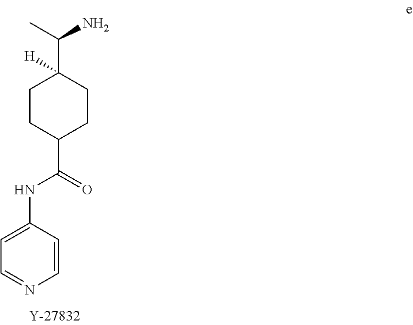

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 or ROCK2 and have much lower activity against PKA, PKG, PKC, and MLCK.

A particularly preferred ROCK inhibitor is fasudil. Fasudil may exist as a free base or salt and may be in the form of a hydrate, such as a hemihydrate.

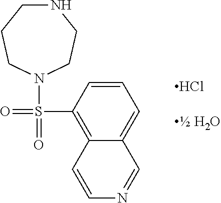

Hexahydro-1-(5-isoquinolinesulfonyl)-1H-1,4-diazepine monohydrochloride hemihydrate

Fasudil is a selective inhibitor of protein kinases, such as ROCK, PKC and MLCK and treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about minutes, but it is substantially converted in vivo to its 1-hydroxy (M3) metabolite. M3 has similar, but non-identical, effects to its fasudil parent molecule in vasodilation mediated by MLCK inhibition, with slightly enhanced activity and a half-life of about 8 hours (Shibuya 2001). It is not known what is the pharmacological contribution of the parent versus the metabolite in vivo, especially regarding the activity on ROCK, the primary target of therapy according to the invention, rather than MLCK. M3 exists as two tautomers, depicted below:

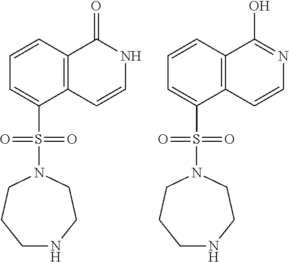

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

### Pharmaceutical Compositions

Pharmaceutical compositions of ROCK inhibitors usable in the are generally oral and may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Methods of Treatment

The invention contemplates treating subjects with age-related cognitive decline. Subjects with age-related cognitive decline will often have deficiencies in processing speed, which is the rate at which cognitive and motor functions are performed. Slowing processing speed can especially affect cognitive functions related to verbal fluency. The inventive methods specifically contemplate treating subjects with reduced processing speed and improving processing speed in such individuals. Processing speed deficits can be assessed using standard tools, like the Symbol Digit Modalities Test and, increasingly, using computerized tests like Processing Speed Test for the iPad®.

Subjects with age-related cognitive decline may also have reduced attention, which is the ability to concentrate and focus. Auditory attention span (immediate or short-term memory) declines slightly with age. Immediate memory is measured by repetition of a string of digits. Declines in selective and divided attention are more significant with age. Selective attention refers to the ability to focus on specific information to the exclusion of irrelevant information. Divided attention is the ability to focus on multiple tasks simultaneously. Related to attention, there is an age-related decline in working memory and specifically the ability to briefly remember something while simultaneously manipulating the information being remembered.

Deficits in memory are very common in subjects with age-related cognitive decline. These may be related to slowed processing speed, reduced selective attention, and decreased use of strategies to improve learning and memory. Memory deficits addressable by the invention may involve declarative memory. Declarative or explicit memory is the conscious recollection of facts and events. Declarative memory includes semantic and episodic memory. Semantic memory involves facts, ideas, meaning, concepts, language usage, and practical knowledge. Episodic memory (also known as autobiographical memory) is memory for personally experienced events that occur at a specific place and time. It can be measured by memory of stories, word lists, or figures. While declines in semantic and episodic memory occur with normal aging, the timing of these declines is different. Episodic memory shows lifelong declines while semantic memory shows late life decline and both episodic and semantic memory deficits are treatable according to the invention.

Nondeclarative (implicit) memory is the other major type of memory and is outside a person's awareness. The classic examples of procedural memory include remembering how to tie a shoe and how to ride a bicycle. Nondeclarative memory remains unchanged with age and is not among the deficits treatable according to the invention.

In addition to types, memory can also be broken down into different stages: acquisition, retention and retrieval. The rate of memory acquisition or the ability to put new things into memory declines with age and is among the deficits treatable according to the invention. Similarly, memory retrieval (the ability to access information) is impaired with age and among the deficits treatable by the invention. Retention of memory, however, is unaffected by advancing age and improved memory retention is not a feature of the invention.

Language deficits in general are not addressable by the invention, except for visual confrontation naming. Visual confrontation naming refers to name an object upon seeing it and this ability declines with age, especially in those over 70 years old. Thus, the invention contemplates improving visual confrontation naming in subjects with age-related cognitive decline.

Some visuospatial dimensions of cognition are addressable by the current invention. Visuospatial function relates the ability to understanding space. These notably treatable in accordance with the invention include deficiencies in visual construction skills, which is ability take individual parts of something and make a coherent whole from them. like assembling a piece of furniture. Visuospatial abilities like recognizing familiar objects or faces and appreciating the physical location of objects generally do not decline solely with age and so are not comprehended by the inventive methods.

Certain deficits in executive functioning in subjects experiencing age-related cognitive decline are also addressable by the inventive methods. Executive functioning refers a person's ability to engage in independent, appropriate, purposeful, and self-serving behavior, including the ability to self-monitor, plan, organize, reason, be mentally flexible, and problem-solve. Deficits treatable using the inventive methods include those in concept formation, abstraction, and mental flexibility, response inhibition, executive abilities requiring a speeded motor component, inductive reasoning (both mathematical and verbal), and reasoning with unfamiliar information, especially in subjects at least age 70. Response inhibition is inhibiting an automatic response in favor of a novel response. Deficits in the ability to comprehend similarities, describe the meaning of proverbs, and reason about familiar information, are not within the scope of the invention.

In addition to changes in cognition, the invention contemplates treating certain pathological changes related to aging and age-related cognitive decline. These include gross structural alterations in brain architecture, like loss of volume in certain regions, as well as a reduction or stabilization of age-related pathologies, like the deposition of beta-amyloid plaques.

The invention specifically contemplates reducing the rate of decline in the volume of both gray matter and white matter regions of the brain. In some cases, these volume changes are due to atrophy, reflecting cell death, but in other cases, it is due to a compaction of the neural cells—neurons, for example may shrink by reducing the number dendrites. Thus, the invention relates to increasing cell volume, number and dendrite number and density.

The invention should be understood to halt or reverse losses in gray matter volume, especially in the prefrontal cortex, the temporal lobes and particularly in the hippocampus. Mitigating loss of volume in the entorhinal cortex, however, is not a feature of the invention.

Beta-amyloid plaques, though better known for their role in Alzheimer's disease (AD), are increasingly understood to be part of the normal aging process, contributing to gray matter volume loss. Using positron emission tomography (PET) to identify beta-amyloid plaques, it has been found in the cortex of up to 20-30% of normal adults. High levels of beta-amyloid appear to be associated with decreased hippocampal volumes and impaired episodic memory in normal, non-AD subjects. The inventive methods further involve reducing the amount of or the rate of accumulation of beta-amyloid plaques in subjects experiencing age-related cognitive decline. Such methods are particularly helpful in stabilizing or reversing decreases in hippocampal volume and improving or stabilizing episodic memory.

The methods of the invention have an even more significant effect on white matter volume loss with age. White matter deterioration, especially white matter, are associated with loss of executive function and so aspects of the invention that reduce white matter deterioration generally also improve executive function and vice versa. The methods of the invention particularly mitigate shrinkage in the precentral gyrus, gyms rectus, corpus callosum, parahippocampal white matter.

Diagnosis of age-related cognitive decline is differential by eliminating pathological processes, most commonly AD. This can be done using imaging and measuring biomarkers in cerebrospinal fluid (CSF). The most widely used CSF biomarkers for Alzheimer's disease measure certain proteins: beta-amyloid 42 (the major component of amyloid plaques in the brain), tau, and phospho-tau (major components of tau tangles in the brain). In Alzheimer's disease, beta-amyloid 42 levels in CSF are low, and tau and phospho-tau levels are high, compared with levels in people without Alzheimer's.

Imaging is as useful tool in diagnosing age-related cognitive decline, in particular computerized tomography (CT), magnetic resonance imaging (MRI) and positron emission spectroscopy (PET). These tools can be sued to look at overall brain volume and can be used to detect the regional losses in volume that are characteristic of age-related cognitive decline. Automated tools are increasingly available that can perform these functions.

Fluorodeoxyglucose (FDG) PET scans measure glucose use in the brain. Glucose, a type of sugar, is the primary source of energy for cells. Studies show that people with dementia often have abnormal patterns of decreased glucose use in specific areas of the brain. An FDG PET scan can show a pattern that may support a diagnosis of dementia versus age-related cognitive decline.

Amyloid PET scans measure abnormal deposits of beta-amyloid. While amyloid plaques also occur in the normal aging brain, higher levels of beta-amyloid are consistent with Alzheimer's disease. Several tracers may be used for amyloid PET scans, including florbetapir, flutemetamol, florbetaben, Pittsburgh compound B and NAV4694.

In accordance with the treatment methods of the present invention, administering a therapeutically effective amount of a ROCK inhibitor or a pharmaceutically acceptable salt thereof one or more times a day. The lowest therapeutically effective amount of fasudil, for example, is 70 mg per day, generally administered in 2 to 3 equal portions to obtain the full daily dose. The highest therapeutically effective dose may be determined empirically as the highest dose that remains effective in alleviating one or more cognitive decline-related signs or symptoms, but does not induce an unacceptable level or adverse events. Fasudil, for example, generally will not be administered in a daily dose exceeding 180 mg. One preferred dosing regimen involves the treatment with 25, 30, 40 or 60 mg of fasudil hydrochloride hemihydrate three times per day using an immediate-release formulation, for a total daily dose of 75-180 mg. Preferred dosing exceeds a daily dose of 70 mg, with most preferred ranges for daily dosing being 70 mg to 140 mg administered in three equal amounts during the day. Other preferred daily doses will range from 90 mg to 180 mg per day or 80 mg to 150 mg per day. A further dosing regimen involves the treatment with, 35 to 90 mg of fasudil hydrochloride hemihydrate only two times per day using an immediate-release formulation, for a total daily dose of 70-180 mg. Generally, an oral daily dose of 70-75 mg will the minimum required to see a treatment effect. At more than 180 mg per day given orally, kidney function begins to be affected and higher dosing in most patients will not be warranted. Above 240 mg per day, kidney effects of the drug are generally unacceptable. Based on ROCK inhibitory activity, one skilled in the art can readily extrapolate the provided dosing ranges for fasudil to other ROCK inhibitors.

The treatment methods of the present invention, while contemplating various routes of administration, are particularly suited to oral administration. Thus, it will be understood that an effective amount of a ROCK inhibitor or a pharmaceutically acceptable salt thereof preferably is administered orally one or more times orally per day and an effective amount may range from the lowest therapeutically effective amount of fasudil, which is 70 mg per day. Generally, it will be administered orally in 2 to 3 equal portions to obtain the full daily dose. The daily oral dose of fasudil, for example, generally will not exceed 180 mg. One preferred dosing regimen involves the treatment with 25, 30, 40 or 60 mg of fasudil hydrochloride hemihydrate three times per day orally using an immediate-release formulation, for a total daily dose of 75-180 mg. Preferred dosing exceeds an oral daily dose of 70 mg, with most preferred ranges for daily dosing being 70 mg to 140 mg administered in three equal amounts orally during the day. Other preferred daily doses will range from 90 mg to 180 mg per day or 80 mg to 150 mg orally per day. A further dosing regimen involves the treatment with, 35 to 90 mg of fasudil hydrochloride hemihydrate only two times per day using an immediate-release oral formulation, for a total daily dose of 70-180 mg. Generally, an oral daily dose of 70-75 mg will the minimum required to see a treatment effect. At more than 180 mg per day given orally, kidney function begins to be affected and higher dosing in most patients will not be warranted. Above 240 mg per day orally, kidney effects of the drug are generally unacceptable. Based on ROCK inhibitory activity, one skilled in the art can readily extrapolate the provided dosing ranges for fasudil to other ROCK inhibitors.

Certain patient sub-populations, such as renally impaired patients and/or older patients (e.g., 65 or older) may need lower doses or extended release formulations instead of immediate release formulations. Fasudil hydrochloride hemihydrate may have higher steady-state concentrations when given at usual doses to patients with renal disease and lower doses to lower the Cmax or delay the time to Cmax (increase the Tmax) may be required.

Renal dysfunction occurs with age and as the result of numerous disorders, including liver cirrhosis, chronic kidney disease, acute kidney injury (for example, due to administering a contrast agent), diabetes (Type 1 or Type 2), autoimmune diseases (such as lupus and IgA nephropathy), genetic diseases (such as polycystic kidney disease), nephrotic syndrome, urinary tract problems (from conditions such as enlarged prostate, kidney stones and some cancers), heart attack, illegal drug use and drug abuse, ischemic kidney conditions, urinary tract problems, high blood pressure, glomerulonephritis, interstitial nephritis, vesicoureteral, pyelonephritis, sepsis. Kidney dysfunction may occur in other diseases and syndromes, including non-kidney-related diseases that may occur along with kidney dysfunction, for example pulmonary artery hypertension, heart failure, and cardiomyopathies, among others.

Kidney function is most often assessed using serum (and/or urine) creatinine. Creatinine is a breakdown product of creatine phosphate in muscle cells and it is produced at a constant rate. It is excreted by the kidneys unchanged, principally through glomerular filtration. Accordingly, elevated serum creatinine is a marker for kidney dysfunction and it is used to estimate glomerular filtration rate.

Normal levels of creatinine in the blood are approximately 0.6 to 1.2 mg/dL in adult males and 0.5 to 1.1 mg/dL in adult females. When creatinine levels exceed these figures, the subject has renal dysfunction, and is, therefore, treatable according to the invention. Mild renal impairment/dysfunction occurs in the range of 1.2 mg/dL to 1.5 mg/dL. Moderate renal impairment/dysfunction is considered to occur at creatinine levels exceeding 1.5 mg/dL. Severe renal impairment, which includes what is considered to be renal failure, is defined as a serum creatinine level of ≥2.0 mg/dL or the use of renal replacement therapy (such as dialysis). Treating subjects with mild, moderate and severe renal impairment is specifically contemplated.

As indicated, creatinine levels are considered to be a surrogate for glomerular filtration rate (GFR) and serum creatinine levels alone may be used to estimate glomerular filtration rate using the Cockroft-Gault equation.

According to the National Kidney Foundation, the following GFRs indicate the varying levels of renal function:

| GFR (ml/min/1.73 m²) | Renal Function |
|---|---|
| ≥90 | Normal or high |
| 60-89 | Mildly decreased |
| 45-59 | Mildly to moderately decreased |
| 30-44 | Moderately to severely decreased |
| 15-29 | Severely decreased |
| <15 | Kidney failure |

In general, creatinine clearance (estimated glomerular filtration rate) may be derived directly from serum creatinine using the Cockroft-Gault equation:

creatinine clearance=(((140−age in years)×(wt in kg))×1.23)/(serum creatinine in μmol/L)

For women the result of the calculation is multiplied by 0.85.

Empirically measured creatinine clearance may also be used directly as an estimate of glomerular filtration rate by looking at serum creatinine and urine creatinine levels. Specifically, urine is collected over 24 hours and the following equation is applied to ascertain creatinine clearance:

Creatinine Clearance(mL/min)=Urine Creatinine Concentration(mg/mL)*24 hour urine volume(mL)/Plasma Creatinine Concentration(mg/mL)*24 hour*60 minutes

In one embodiment, dose of fasudil for mild to moderate renal impairment is reduced to 50-80 mg per day. In another embodiment, the dose of fasudil is not reduced but is administered one time per day in an extended release dosage form.

In another embodiment, the dose is not reduced for mild to moderate renal impairment.

In one embodiment, the dose of fasudil is reduced to 30-45 for severe renal impairment. In another embodiment, the dose of fasudil is not reduced but is instead administered one time per day in an extended release dosage form.

In a further embodiment, the dose is reduced where serum creatinine (SCr)>2 and/or an increase in SCr>1.5× from baseline, and/or a decrease in eGFR>25% from baseline.

Patient size is an important factor to consider when using creatinine-based estimates of renal function. The units of drug clearance are volume/time (mL/min), whereas the units of estimated GFR for chronic renal disease are volume/time/standard size (mL/min/1.73 m²). Generally, doses may be adjusted down (e.g., 40-50 mg per day) for smaller patients and up for larger (e.g., 120 mg per day) for obese patients. A smaller male would be about 160 pounds or less. A smaller female patient would weigh about 130 pounds or less. Patients having a Body Mass Index of 30 and higher is considered obese.

In addition, older patients may need a lower dose at initiation, with a gradual increase to the recommended dose after days or weeks. In another embodiment, older patients may need lower doses for the duration of treatment. The aged population includes the “young old” who are 65-74, the “old old” who are 75-84 and the “frail elderly” who are 85 and older. For example, a starting dose of 30 mg per day for two weeks, followed by 60 mg per day for 4 weeks, then by 90 mg per day. Titration may even be warranted up to about 120 mg per day.

Another embodiment involves the treatment with 60-120 mg of fasudil hydrochloride hemihydrate once per day in an extended release dosage form. Treatment with an extended release total daily dose of 90 mg fasudil hydrochloride hemihydrate once per day is preferred. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

Another embodiment involves the treatment with 60-120 mg of fasudil hydrochloride hemihydrate once per day in an extended release dosage form. Treatment with an extended release total daily dose of 90 mg fasudil hydrochloride hemihydrate is preferred.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment.

In diagnosing age-related cognitive decline, the cognitive deficit may be detected using a number of different tests. Generally useful tests include the Montreal Cognitive Assessment (MoCA) and the Mini-Mental State Exam (MMSE).

The MoCA is a one-page 30-point test that takes approximately 10 minutes to administer. It measures the following cognitive domains. Short-term memory where the subject learns 5 nouns in two trials and then is asked to recall them after 5 minutes. Visuospatial abilities are assessed using a clock-drawing task (3 points) and a three-dimensional cube copy (1 point). Executive function is assessed using an alternation task (variation the trail-making task) (1 point), a phonemic fluency task (1 point), and a verbal abstraction task (2 points). Attention, concentration, and working memory are evaluated using a sustained attention task (1 point), a serial subtraction task (3 points), and digits forward and backward (1 point each). Language is assessed using a three-item confrontation naming task (lion, camel, rhinoceros; 3 points), repetition of two syntactically complex sentences (2 points), and the phonemic fluency task, mentioned above. Abstract reasoning is assessed using a describe the similarity task (2 points). Finally, orientation to time and place is evaluated by asking the subject for the date and the city in which the test is occurring (6 points). Scores range between 0 and 30. A score of 26 -30 is considered normal and a score of less than 26 is considered cognitively impaired. Accordingly, subjects treatable according to the invention preferably have a MoCA score of 25 or less. The MoCA is believed by certain people to be very difficult, especially the last five questions.

The MMSE, is described fully in Folstein (1975, 1987 and 2007) and is very similar to the MoCA. An MMSE score of 24-30 indicates no cognitive impairment and a score or less is considered to be cognitively impaired. Accordingly, subjects treatable according to the invention preferably have a MMSE score of 23 or less.

In addition, once a cognitive deficit is identified by administering a general test, more specific tests, such as those in the following table, may be administered in order to identify the defective cognitive domain(s) and to quantify any such deficiency or deficiencies.

### Common Cognitive Tests

| Test | Description |
|---|---|
| Processing Speed | |
| Pattern Comparison Processing | Determine whether a set of two pictures placed side by |
| Speed Test | side are the same for 90 seconds. |
| Digit Symbol Substitution Test | Timed measure consisting of nine digit symbol pairs; |
| | participant matches the correct symbol to a list of digits. |
| Sustained attention | |
| Connors Continuous Performance | Measure selective response to target inputs while doing a |
| Test | repetitive task-for example, respond only when they will |
| | see or hear the number 1, but not the number 2. |
| Selective attention | |
| Target Letter Search | Search a visual display for a target, such as a letter, that is |
| | surrounded by other, non-target letters. |
| Stroop Test | Name the color of ink in which another color word is |
| | printed (e.g., the word “red” printed in the color green). |
| Episodic memory | |
| Picture Sequence Memory Test | Recall series of 6 to 18 illustrated objects and activities |
| | presented in a certain order. |
| Working memory | |
| General | Common tests include giving participants a list of items |
| | (letters, numbers, words) and asking them to repeat the |
| | items back in order. |
| List Sorting Test | Participants see pictures and written names of foods and |
| | animals and hear audio names. The participant recites |
| | them back in order of size from smallest to largest, first |
| | within a single category (e.g., animals) and then in two |
| | categories (e.g., foods, then animals). |
| Semantic memory | |
| Category Fluency Test | Name all of the items they can in a category (e.g., animals) |
| | in 1 minute. |
| Boston Naming Test | Subject sees a series of pictures and must name each |
| | picture within 20 seconds |
| Executive function | |
| Wisconsin Card Sorting Test | Subject matches two sets of cards according to some |
| | characteristic, but the participant is not told the rules- |
| | they learn through feedback. After a fixed number of |
| | correct matches, the rules are changed without notice, and |
| | the new rule must be learned. |
| Trail Making Test | Subject connects 25 consecutive numbers or alternating |
| | numbers and letters in ascending order. |
| Reasoning | |
| Letter Sets Test | consists of five sets of four letters. The participant has to |
| | decide which one of the five sets is dissimilar in the sense |
| | that it does not follow a rule used to generate the other four |
| | sets. Tests of inference are used to measure the ability to |
| | reason and draw conclusions from information presented |
| | in statements. |
| Language | |
| General | There are a variety of tests, depending on the particular |
| | aspect of language being assessed. These include a word- |
| | by-word reading paradigm to assess comprehension of |
| | sentences (e.g., Kemtes and Kemper, 1997), word-by- |
| | word reading time (e.g., Stine, 1990) or recall of text |
| | (Stine-Morrow et al., 1996). |
| Token Test | Subject presented with tokens that vary in shape color and |
| | size and must follow commands related to identifying |
| | them (e.g., touch the circle). |
| Boston Naming Test | Confrontational naming test-subject shown objects to |
| | name in certain period of time. |
| Category Fluency Test | Subject is asked to name items in a category, like words |
| | beginning with F, A and S, or as many animals as possible |
| | in a particular time. |
| Boston Diagnostic Aphasia | A testing battery that evaluates language skills based on |
| Examination | perceptual modalities (auditory, visual, and gestural), |
| | processing functions (comprehension, analysis, problem- |
| | solving), and response modalities (writing, articulation, |
| | and manipulation). |
| Spatial ability | |
| Paper Folding Test | Decide which pieces of paper with holes punched in them |
| | is the same by looking at them folded and unfolded. |
| Mental Rotations Test | Compare several three-dimensional objects that are |
| | rotated differently and state whether they are the same or |
| | mirror images. |
| Block Design Test | Subtest of the Wechsler Adult Intelligence Scale (WAIS). |
| | Arrange a set of 3-dimensional blocks to match a 2- |
| | dimensional drawing using color cues within a specific |
| | time limit. |
| Intelligence | |
| Wechsler Adult Intelligence Scale | Consists of 10 core subtests and 5 supplemental subtests. |
| | The core subtests provide the intelligence quotient, which |
| | is derived from four index scores: Verbal Comprehension |
| | Index, Perceptual Reasoning Index, Working Memory |
| | Index, and Processing Speed Index. |

Treatment using the inventive methods generally result in improved cognitive functioning on general tests of cognition like the MoCA or the MMSE and/or on cognitive tests directed to one or more cognitive domains. Scores or times (as appropriate for the test) will generally improve by a minimum of 10% with treatment as compared to without treatment. Thus, MoCA and/or MMSE scores will improve by at least 2 points and generally by at least 3 points with drug treatment. In addition, subjects treated with drug will show reduced rate of cognitive decline than those who are not treated, as measure by the MoCA, the MMSE and/or one or more specific cognitive tests. In some embodiments, improvements in scores or times will be a minimum of 20% on treatment as compared to without treatment. In a preferred embodiment, treatment of a patient with fasudil reduces or reverses the progression of age-related cognitive decline by one or more of the foregoing cognitive measures. The methods of the invention also contemplate administering ROCK inhibitors with other compounds used to improve cognition. They may be administered in combination, a single dosage form, in a common dosing regimen or administered to the same patient at different times of the day using different dosing regimens.

### LIST OF REFERENCES

Chen M, Liu A, Ouyang Y, Huang Y, Chao X, Pi R. 2013. Fasudil and its analogs: a new powerful weapon in the long war against central nervous system disorders? Expert Opin Investig Drugs. 22:537-50.
Feng Y, LoGrasso P, Defert O, Li R, Rho Kinase (ROCK) Inhibitors and Their Therapeutic Potential. J Med Chem. 2016; 59*6): 2269-2300.
Folstein M F, Folstein S E, McHugh P R. “Mini-mental state”: a practical method for grading the cognitive state of patients for the clinician. J Psychiatr Res. 1975; 12:189-198.
Huentelman M J, Stephan D A, Talboom J, Corneveaux J J, Reiman D M, Gerber J D, Barnes, C A, Alexander G E, Reiman E M, Bimonte-Nelson, H A, Peripheral Delivery of a ROCK Inhibitor Improves Learning and Working Memory. Behav Neurosci. 2009; 123(1): 218-223.
Jacobs M, Hayakawa K, Swenson L, Bellon S, Fleming M, Taslimi P, Doran J, The structure of dimeric ROCK I reveals the mechanism for ligand selectivity. J Biol Chem. 2006; 281(1): 260-68.
Nakagawa O, Fukisawa K, Ishizaki T, Saito Y, Nakao K, Narumiya S, ROCK-I and ROCK-II, two isoforms of Rho-associated coiled-coil forming protein serine/threonine kinase in mice. FEBS Lett. 1996 Aug. 26; 392(2):189-93.
Rovner B W, Folstein M F. Mini-mental state exam in clinical practice. Hosp Pract. 1987; 22(1A):99, 103, 106, 110.
Salthouse T A, When does age-related cognitive decline begin? Neurobiol Aging. 2009; 30(4): 507-514.
Shibuya M, Asano T, Sasaki Y. 2001. Effect of Fasudil HCl, a protein kinase inhibitor, on cerebral vasospasm. Acta Neurochir Suppl. 77:201-4.
Uehata M, Ishizaki T, Satoh H, Ono T, Kawahara T, Morishita T, Tamakawa H, Yamagami K, Maekawa M, Narumiya S, Calcium sensitization of smooth muscle mediated by a Rho-associated protein kinase in hypertension. Nature. 1997 Oct. 30; 389(6654):990-4.
Yamaguchi H, Miwa. Y, Kasa M, Kitano K, Amano M, Kaibuchi K, Hakoshima T, Structural basis for induced-fit binding of Rho-kinase to the inhibitor Y-27632. J Biochem. 2006 September; 140(3):305-11.

The disclosure of each reference set forth herein is incorporated herein by reference in its entirety.

1. A method of reducing the rate of age-related cognitive decline, comprising administering to a patient experiencing age-related cognitive decline an effective amount of a rho kinase inhibitor, wherein the rho kinase inhibitor is administered in a dose of at least 70 mg per day.

2. The method according to claim 1, wherein processing speed in the patient experiencing age-related cognitive decline is enhanced upon administration of an effective amount of the rho kinase inhibitor.

3. The method according to claim 2 wherein the patient displays reduced verbal fluency.

4. The method according to claim 1, wherein attention in the patient experiencing age-related cognitive decline is increased upon administration of an effective amount of the rho kinase inhibitor.

5. The method according to claim 1, wherein memory in the patient experiencing age-related cognitive decline is improved upon administration of an effective amount of the rho kinase inhibitor.

6. The method according to claim 5 wherein the patient has a deficit in declarative memory.

7. The method according to claim 6 wherein the patient has a deficit in semantic memory.

8. The method according to claim 7 wherein the patient is less than 70 years old.

9. The method according to claim 5, wherein the patient is suffering from a reduced rate of memory acquisition.

10. The method according to claim 5, wherein the patient is suffering from a reduced ability to retrieve memories.

11. A method of improving language in a patient experiencing age-related cognitive decline, comprising administering to the patient an effective amount of a rho kinase inhibitor.

12. The method according to claim 11, wherein the patient shows a deficit in visual confrontation naming.

13. The method according to claim 12, wherein the patient is at least 70 years old.

14. The method according to claim 11, wherein the patient shows a deficit in verbal fluency.

15. The method according to claim 11, wherein visual construction skills are improved in the patient experiencing age-related cognitive decline upon administration of the rho kinase inhibitor.

16. The method according to claim 1, wherein executive function is improved in the patient experiencing age-related cognitive decline upon administration of the rho kinase inhibitor.

17. The method according to claim 16, wherein the patient has a deficiency in one or more of the following abilities: concept formation, abstraction, mental flexibility, response inhibition, speed-motor executive functions, inductive reasoning and reasoning with unfamiliar information.

18. The method according to claim 16, wherein the patient is at least 70 years old.

19. The method according to claim 1 wherein the rho kinase inhibitor is an isoquinoline derivative.

20. The method according to claim 19 wherein the isoquinoline derivative is fasudil, a salt, or a derivative thereof.

21. The method according to claim 19 where the treatment continues for at least 6 months.

22. (canceled)

23. The method according to claim 19, wherein the dose is administered in three equal portions throughout the day.

24. The method according to claim 23, wherein the total daily dose is between 70 mg and 180 mg.
